(19) United States
(12) Patent Application Publication
(10) Pub. No.: US 2004/0215310 A1
(43) Pub. Date: Oct. 28, 2004

(54) Stent and delivery method for applying RF energy to a pulmonary vein and the atrial wall around its ostium to eliminate atrial fibrillation while preventing stenosis of the pulmonary vein thereafter

(76) Inventor: Omar Amirana, San Francisco, CA (US)

Correspondence Address: VIERRA MAGEN MARCUS HARMON & DENIRO LLP, 685 MARKET STREET, SUITE 540, SAN FRANCISCO, CA, 94105, US

(21) Appl. No.: 10/346,232

(22) Filed: Jan. 17, 2003

Related U.S. Application Data
(60) Provisional application No. 60/350,248, filed on Jan. 17, 2002.

(51) Int. Cl. A61F 2/02

(52) U.S. Cl. 623/1.11

(57) ABSTRACT

A stent and delivery method are disclosed for positioning the stent within and around one or more pulmonary veins to deliver ablative (RF) energy to the vein and surrounding atrial wall tissue to eliminate and prevent atrial fibrillation via RF ablation through said stent. After the procedure is completed, said stent remains in position to prevent stenosis of the pulmonary vein.

### CLAIM OF PRIORITY

This application claims the benefit of U.S. Provisional Application No. 60/350,248 filed Jan. 17, 2002.

### BACKGROUND OF THE INVENTION

1. Field of the Invention

The present invention relates in general to the treatment of atrial fibrillation, and in particular to a method and apparatus comprising a stent for delivering radiofrequency energy to the pulmonary vein, or the pulmonary vein and left atrium around the vein ostium, to eliminate atrial fibrillation, and for thereafter preventing stenosis of the pulmonary vein.

2. Description of the Related Art

Atrial fibrillation (AF) is the most common cardiac arrhythmia, affecting over two million people in the United States alone. One significant source of paroxysmal AF has been shown to be one or more of the pulmonary veins. The etiology of AF is poorly understood and it is generally agreed to be a disease of age with a broad spectrum of causes, severity and symptoms. Many believe AF is due to electrical changes in the atrial tissue due to atrial fibrosis. During fetal maturation, the pulmonary veins grow out of the atrium to the lungs to allow deoxygenated blood to be carried back to from the heart. Some researchers speculate that, as the pulmonary veins grow out of the atrium, bands or sections of cardiac cells may be pulled into the ostia of the pulmonary veins or beyond. It has been found that these cardiac cells have electrical automaticity, and may rapidly and randomly fire to trigger ectopic heart beats that interfere with or destroy the normal sinus rhythm of the heart.

Pharmacological treatment of AF includes antiarrhythmics and drugs such as sotalol, propafenone and amidodarone to preserve normal sinus rhythm. However, such treatments are palliative and do not address the underlying causes of AF. Moreover, individual patients may have negative or intolerant reactions to pharmacological treatments.

It is also known to treat AF by surgical procedures. One well-known technique is the maze procedure where the chest is opened, and a series of surgical incisions are made on the atrium to form lesions which segment the atrium into sections too small to allow propagation of the electrical wavelets generated during AF. However, as with any cardiac surgery, these procedures are extremely invasive, costly and are associated with a high risk of morbidity and mortality.

Recently, techniques have been developed to treat AF by minimally invasive catheter ablation procedures. One such procedure is described in Pappone, et al., Circumferential Radiofrequency Ablation of Pulmonary Vein Ostia: A New Anatomic Approach for Curing Atrial Fibrillation, Circulation, 2000; 102:2619-2628. As set forth in that and similar publications, a catheter is introduced into the pulmonary vein from the left atrium, which catheter includes an electrode at its distal end capable of delivering radiofrequency (RF) energy to cardiac tissue to controllably scar or ablate it. The electrode is manipulated by controls in the catheter handle to apply the RF energy around the circumference of the pulmonary vein ostium to form a circumferential scar or lesion that electrically isolates the cardiac cells in the pulmonary vein from the rest of the heart, preventing them from interfering with the normal sinus rhythm of the heart.

While RF catheter ablation presents a marked advance over traditional surgical treatment of AF, RF catheter ablation presents several drawbacks. One significant drawback is the risk of stenosis of the pulmonary vein after the procedure due to the circumferential scar tissue or muscular contraction secondary to trauma. If not caught, pulmonary vein stenosis can lead to pulmonary hypertension, right-sided cardiac failure and even death.

A further disadvantage of RF catheter ablation is the difficulty in manipulating the distal end of the catheter to properly form the circumferential lesion. Additionally, the walls of the pulmonary veins are susceptible to perforation, and care must be taken while manipulating the distal end of the catheter not to tear or damage the veins. Moreover, there may be several focal initiation points of AF in a single pulmonary vein and it is difficult to ensure that the formed lesion encompasses all of the ectopic focal points. Furthermore, even where the lesion is properly formed, the procedure takes a significant amount of time. Formation of a single circumferential lesion may take on the order of 2 to 8 hours, and it may be that more than one pulmonary vein includes ectopic cardiac cells, thus requiring the formation of the circumferential lesions in multiple pulmonary veins.

A still further disadvantage to RF catheter ablation is the potential damage to surrounding tissue. Damage to adjacent extracardiac structures such as bronchioles, the right pulmonary artery, phrenic nerve and lung tissue have been documented. Perhaps more significantly, the applied RF energy can cause thrombosis and/or embolism in the blood flowing through the pulmonary vein. Migration of a blood clot or embolism to vital organs can cause a stroke or death of the patient.

### SUMMARY OF THE INVENTION

Embodiments of the present invention relate to a stent and delivery method for positioning the stent within and around one or more pulmonary veins to eliminate and prevent atrial fibrillation via RF ablation through the stent. After the RF ablation procedure through the stent is completed, the stent remains in position to prevent stenosis of the pulmonary vein. The stent may be deployed from the distal end of a catheter positioned within a pulmonary vein from the left atrium of the heart. In one embodiment, the stent may be a self-expanding helical coil formed of a shape memory metal or alloy. As the stent is deployed from the distal tip of the catheter, radial forces within the stent cause it to expand radially into contact with the walls of the pulmonary vein and left atrium around the pulmonary vein ostium.

In embodiments of the present invention, the proximal and distal ends of the stent are provided in such a way as to form closed circumferential lesions in pulmonary vein and around its ostium upon application of RF energy to the stent. The distal end of the stent lies within the pulmonary vein, while the proximal end of the stent is provided to rest in the left atrium, outside of the pulmonary vein. Toward this end, the proximal end of the stent has a larger circumference than the adjacent distal sections in a radially expanded condition. Thus, upon being fully deployed from the catheter, the size and inherent bias of the loops at the proximal end of the stent position the proximal end of stent snugly against the wall of the left atrium, circumjacent about the pulmonary vein ostium.

Tissue in the pulmonary vein and left atrium surrounding the vein are ablated by the application of energy, such as radiofrequency energy, through the stent during or after deployment. While conventional RF catheter ablation techniques are capable of creating a single circular lesion around a pulmonary vein, a stent in accordance with the present invention is capable of creating circular and helical lesions around a circumference of the vein, across a relatively large length of the pulmonary vein, and across a section of the left atrium surrounding and encircling the pulmonary vein ostium. Thus, the present invention offers greater potential to cure AF as compared to conventional treatment methods. Moreover, as the stent naturally expands into the desired position in contact with the pulmonary vein wall and atrium around the vein, the present invention is able to perform the ablation operation in a quicker and easier procedure as compared to the prior art. The stent also remains behind after the catheter is withdrawn to prevent stenosis of the pulmonary vein which may otherwise occur in conventional RF catheter ablation procedures.

As an alternative to a self-expanding stent that is deployed from the distal tip of a catheter, a stent in accordance with the present invention may alternatively be deployed with a conventional balloon catheter. In this embodiment, a stent is removably fastened about an angioplasty balloon, for example by being crimped onto the deflated balloon. The balloon is positioned in the pulmonary vein, at which point the balloon is inflated until the stent lies in firm contact with the wall of the pulmonary vein. Thereafter, the balloon is deflated, leaving the stent in position in contact with the pulmonary vein wall. Once the stent is in contact with the pulmonary vein, RF energy may be applied to and through the stent to ablate the tissue around the circumference and along the length of the stent.

### BRIEF DESCRIPTION OF THE DRAWINGS

The present invention will now be described with reference to the drawings, in which:

FIG. 1 is a perspective view of a catheter and stent device used in accordance with the present invention;

FIG. 2 is a perspective view of a distal end of a stent being deployed within a pulmonary vein in accordance with the present invention;

FIG. 3 is a perspective view of a proximal end of a stent being deployed in the left atrium around the ostium of a pulmonary vein in accordance with the present invention;

FIG. 4 is a side view of the geometry of a stent in accordance with the present invention;

FIG. 5 is a side view of a pushwire and stent according to the present invention;

FIG. 6 is a side view of a pushwire and stent according to an alternative embodiment of the present invention;

FIG. 7 is a side view of a pushwire and stent according to an alternative embodiment of the present invention;

FIG. 8 is a stent according to an alternative embodiment of the present invention;

FIG. 9 is a stent according to a further alternative embodiment of the present invention;

FIG. 10 is a stent according to a still alternative embodiment of the present invention;

FIG. 11 is an alternative catheter for applying a stent according to the present invention; and

FIG. 12 is a further alternative catheter for applying a plurality of stents according to the present invention.

### DETAILED DESCRIPTION

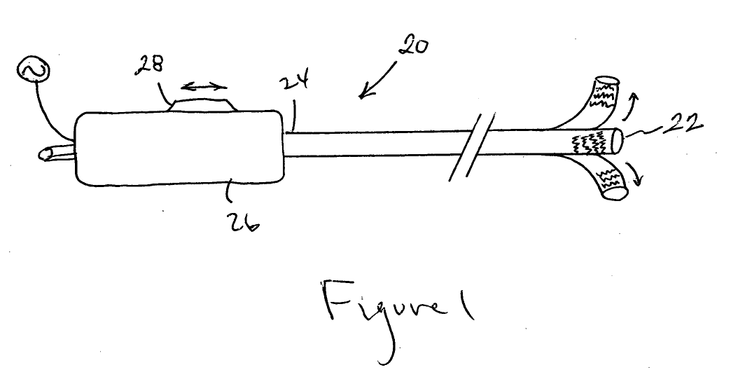

The present invention will now be described with reference to FIGS. 1-12, which in general relate to a stent and delivery method for positioning the stent within the pulmonary vein to prevent or eliminate atrial fibrillation via RF ablation through the stent. The stent remains in position after ablation to prevent stenosis of the pulmonary vein. The present invention may, however, be embodied in many different forms and should not be construed as being limited to the embodiments set forth herein; rather these embodiments are provided so that this disclosure will be thorough and complete and will fully convey the invention to those skilled in the art. Indeed, the invention is intended to cover alternatives, modifications and equivalents of these embodiments, which will be included within the scope and spirit of the invention as defined by the appended claims. Furthermore, in the following detailed description of the present invention, numerous specific details are set forth in order to provide a thorough understanding of the present invention. However, it will be clear to those of ordinary skill in the art that the present invention may be practiced without such specific details. In other instances, well known methods, procedures, components, and circuits have not been described in detail as not to unnecessarily obscure aspects of the present invention.

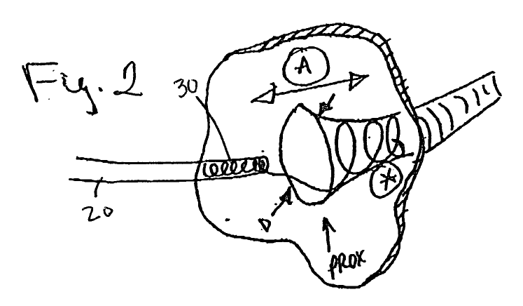

Referring now to FIG. 1, there is shown a catheter 20 including a distal end 22 and a proximal end 24 having a handle 26 and controls 28 for manipulating distal end 22. Distal end 22 includes a stent 30 for deployment at and in a pulmonary vein as explained hereinafter. Catheter 20 may be of known design and has a diameter of 4-12 French. Catheters of other configurations and diameters are contemplated. As is known in the art, distal end 22 of catheter 20 may be positioned at or within a pulmonary vein through a transseptal sheath 32 (FIG. 2) terminating in the left atrium. The sheath 32 is preferably insulated to route RF energy only to the stent and to prevent RF energy delivered to the stent 30 from affecting tissue through which the catheter 20 is passed.

In one embodiment, stent 30 may comprise a self-expanding helical coil formed of a shape memory metal or alloy, such as for example Nitinol (nickel titanium), loaded within the distal end 22 of catheter 20. In such embodiments, distal end 22 may be inserted into a pulmonary vein, and the stent 30 may be deployed as the distal end is withdrawn from the pulmonary vein back into the left atrium. A contrast dye may be injected through a lumen in catheter 20 into the pulmonary vein to allow fluoroscopic visualization of the size and contours of the vein, as well as to ensure proper deployment of the stent 30 as explained hereinafter.

Referring to FIG. 2, as stent 30 is deployed from the distal tip of catheter 20, radial forces within the stent cause it to expand radially into contact with the walls of the pulmonary vein. The distal end of stent 30 may be deployed approximately 0.2cm to 6 cm past the ostium of the pulmonary vein. However, it is understood that the distal end of stent 30 may extend greater or lesser than 0.2cm to 6cm into the pulmonary vein in alternative embodiments.

As shown in FIG. 2, in one embodiment of the present invention, the most distal loops of stent 30 lie in contact with each other (i.e., compressed against each other) when the stent is in an expanded and unbiased condition. When the stent is deployed, the last few loops at the distal end of the coil are inserted so as to remain in contact with each other. This configuration ensures a circumferential lesion in the pulmonary vein at the distal end of the stent 30 upon application of RF energy as explained hereinafter. It is understood that more than merely the last few loops may lie in contact with each other at the distal end of stent 30 upon deployment of the stent in the pulmonary vein in alternative embodiments of the invention. Moreover, it is understood that the distal loops need not lie in contact with each other in an unbiased condition, and/or need not be deployed in contact with each other in alternative embodiments of the invention.

As would be appreciated by those of skill in the art, stent 30 is selected so that the radial forces in an expanded condition are sufficient to anchor the stent in position against the walls of the pulmonary vein and to hold the vein open, but not so high as to perforate or cause damage to the pulmonary vein walls. At sections between the distal and proximal ends of the stent 30, the spacing between adjacent loops in the deployed stent may range from between approximately 1 mm to 20 mm, and optimally around 3 mm to 5 mm. It is understood that the spacing between the loops may be smaller or larger than the above-described range in alternative embodiments.

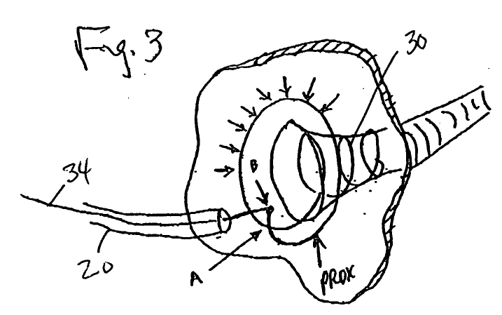

Referring now to FIGS. 3 and 4, the proximal end of stent 30 preferably has a larger circumference than the adjacent distal sections in a radially expanded condition. Additionally, in an expanded and unbiased condition, the proximal loops of stent 30 lie in contact with each other. In an embodiment with such a stent configuration, after the distal end of the stent has been deployed and anchored in the pulmonary vein, the proximal end of the stent is deployed from the delivery catheter in the left atrium, outside of the pulmonary vein. Upon the distal end and middle portions of the stent 30 being anchored, or deep seated, in the pulmonary vein, the remainder of the stent may be deployed in a slightly stretched condition so as to create a tensile force along a central axis of the stent. Thus, as shown in FIG. 3, upon the proximal end being deployed from the catheter 20 in the left atrium, the size of the proximal loops, the inherent bias of the loops, and the tensile forces within the stent cause the proximal end to elastically recoil into contact with the atrial wall surrounding the pulmonary vein ostium, where the proximal end remains.

It is understood that in alternative embodiments, the proximal end of stent 30 may have the same size circumference as other portions of stent 30, in which embodiments, the proximal end of stent 30 would fit entirely within the pulmonary vein. Moreover, it is understood that the proximal loops need not lie in contact with each other in an unbiased condition, and/or need not be deployed in contact with each other in alternative embodiments of the invention.

In embodiments of the invention described above, the proximal and distal ends of stent 30 have loops that lie in contact with each other. A function served by this structure is to create a closed circumferential loop lesion (or scar) in the tissue in contact with and in proximity to the proximal and distal ends of the stent when RF energy is delivered through the stent. It is understood that a closed circumferential loop lesion may be created at the proximal and/or distal ends by stent structures other than those described above. For example, instead of the loops at the proximal and/or distal ends lying in contact with each other, the proximal and/or distal ends may have a discontinuous bend, bringing the end into contact with the next adjacent loop. The discontinuous bend may also be at a position or positions other than the proximal and/or distal ends of the stent 30. Instead of a bend which is discontinuous, the bend may alternatively be gradual. Other structural configurations are contemplated to accomplish the function of creating a closed circumferential loop lesion in the tissue in contact with the proximal and/or distal ends of stent 30.

The catheter 20 of the embodiment shown in FIGS. 2 and 3 further includes a pushwire 34 having a distal end removably affixed to the proximal end of stent 30, and a proximal end protruding from handle 26 in the proximal end of the catheter 20. Pushwire 34 may be manipulated by the cardiologist to move the stent 30 relative to catheter 20 to allow deployment of the stent. While termed a pushwire, it is understood that pushwire 34 may push or pull stent 30 relative to catheter 20.

In embodiments of the present invention, pushwire 34 may also be used to conduct RF energy to the stent 30 to form a circumferential and helical (or other patterned) lesion in the pulmonary vein and left atrial wall around the pulmonary vein ostium. The closed circumferential loop lesion formed by the distal end of the stent and the closed circumferential loop lesion formed by the proximal end of the stent electrically isolate the pulmonary vein from the heart, and prevent ectopic foci in the pulmonary vein and/or the pulmonary vein ostium from triggering ectopic heartbeats. In particular, in embodiments of the invention, the pulmonary vein is electrically isolated from the heart at two distinct locations: inside the pulmonary vein by the distal closed circumferential loop lesion to prevent ectopic foci inside the vein from triggering an ectopic heartbeat; and outside the pulmonary vein by the proximal closed circumferential loop lesion to prevent ectopic foci in the pulmonary vein ostium from triggering an ectopic heartbeat.

It is understood that in alternative embodiments, the closed loop in the stent at the distal or proximal end of the stent may be omitted, so that the stent forms only one circumferential lesion at its distal or proximal end, and helical (or other patterned) lesion adjacent thereto.

Additionally, it is understood that both the closed loop at the distal and proximal ends of the stent may be omitted in alternative embodiments. In these embodiments, even if no closed circumferential loop lesion is formed, the stent 30 may form a helical lesion which is effective in preventing ectopic heartbeat. In particular, in pulmonary vein AF, ectopic electrical impulses travel essentially in a straight linear path from the pulmonary vein to the left atrium where they can interfere with normal sinus heartbeat. By creating a helical lesion in accordance with embodiments of the invention, the ectopic electrical impulses are prevented from traveling in a straight path to the left atrium, and are instead forced to follow a helical path, due to the helical lesion, into the left atrium. This increased path length delays the electrical impulses from reaching the left atrium and as such greatly reduces the possibility of the ectopic impulses interfering with normal sinus rhythm of the heart. The circumferential and/or helical lesions formed in accordance with the present invention are more effective at electrically isolating and/or killing ectopic foci at various locations in and along the pulmonary veins. Thus, the present invention offers greater potential to both prevent and cure AF as compared to conventional treatment methods.

Moreover, where it is difficult and time consuming to form the circumferential lesion using conventional RF catheter ablation techniques, the stent 30 described above is able to ablate tissue in and around the pulmonary vein easily and quickly. In particular, the lesion is more easily formed in comparison to conventional methods because the stent naturally expands into contact with the pulmonary vein wall, and naturally recoils against the atrial wall surrounding the pulmonary vein ostium. Thus, the conventionally difficult procedure of maneuvering the catheter by a cardiologist to form the circumferential lesion is avoided. Moreover, because the stent provides a much larger surface area in contact with the tissue (around the circumference of the stent and along the length of the stent) in comparison to conventional catheter ablations techniques, once RF energy is applied, it is received instantaneously around the entire circumference of the pulmonary vein wall and ostium, and in a pattern along a length of the wall. Thus, the ablative lesions are formed in a fraction of the time in comparison to conventional techniques.

In order to supply RF energy to stent 30, pushwire 34 may be connected at its proximal end to an RF generator (not shown). In one embodiment, RF energy may be supplied from the generator, through the pushwire 34 and stent 30 to the pulmonary vein and atrium. It is understood that the RF energy may be supplied to the stent 30 via a second wire (not shown) in contact with the stent 30, which second wire is separate from the pushwire 34. This second wire may be a guide wire conventionally used to position the catheter in the left atrium. In such an embodiment, pushwire 34 would merely serve to move the stent 30 relative to the catheter 20.

The RF energy is applied to the tissue through the stent 30 in bursts and/or in a constant stream at sufficient duration and intensity to ablate the tissue with which the stent is in contact. It is understood that the RF energy may be applied to ablate tissue while the stent is being deployed and/or after the stent is deployed. Moreover, while RF energy has been described, the present invention may alternatively employ other forms of energy to ablate tissue in the pulmonary vein and atrium. While not exhaustive, additional forms of energy may include electromagnetic energy in bands other than radiofrequency. A large adhesive disposable ground pad may be placed on the patient's thigh or flank (side towards back) and then plugged into the RF generator to complete the circuit.

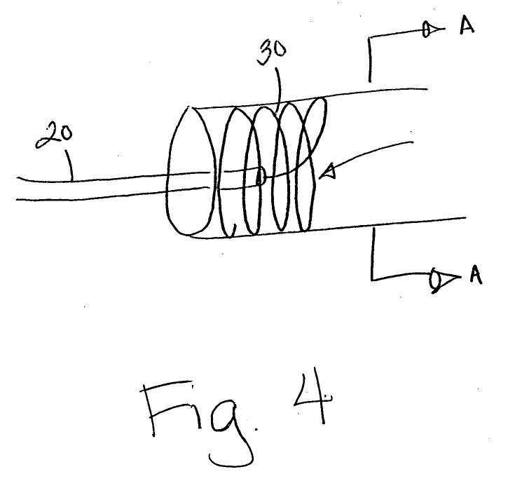

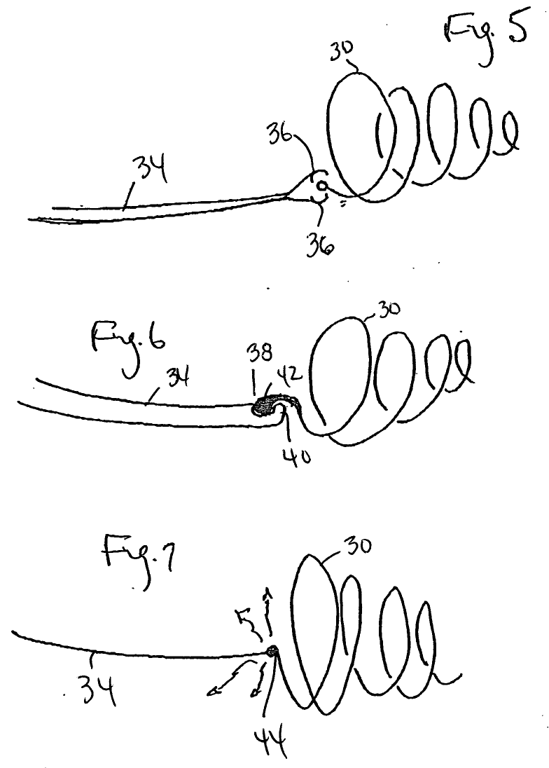

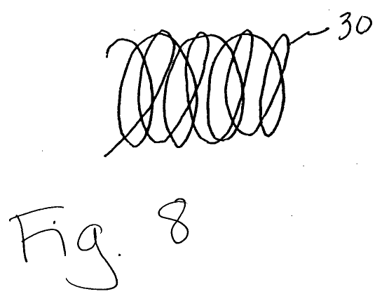

Once the stent is deployed and the tissue has been ablated, the pushwire may be detached from the stent and removed with the catheter. FIGS. 5 through 7 illustrate various mechanisms by which pushwire 34 may be detached from stent. In FIG. 5, pushwire 34 includes a pair of gripping jaws 36 for gripping the proximal end of stent 30, which jaws 36 may be connected to a control 28 through the catheter 20. Upon deployment of the stent 30, the jaws 36 may be opened to release the stent. In FIG. 6, pushwire 34 is affixed to the stent via an interlock 38 comprising a first hook 40 on the pushwire 34 that interlocks with a second hook 42 on the stent 30. Upon deployment of the stent 30 and delivery of RF energy through the stent to ablate the tissue, the first and second hooks may be detached from each other by twisting of the first hook 40, which is connected to a control 28 through the catheter 20. In FIG. 7, the pushwire 34 is affixed to the stent 30 via a meltable, dissolvable or electrolytic junction 44. Once the stent is deployed, the meltable, dissolvable or electrolytic junction 44 may be heated by application of energy through the pushwire to melt the junction and separate the pushwire and stent. It is understood that the melting temperature of junction 44 may be above that required to ablate the tissue in the pulmonary vein and atrium, but below that which would cause damage to the surrounding tissue. It is understood that other known detachment mechanisms may be used to detach pushwire 34 from stent 30.

Stent 30 has been described as a wound helix that expands when deployed from the tip of the catheter 20. It is understood that the self-expanding stent 30 may have other configurations in alternative embodiments. For example, FIG. 8 illustrates a stent 30 having a double reverse helix. Such a stent creates a lattice pattern of quadrangular-shaped ablation lesions on the pulmonary vein wall.

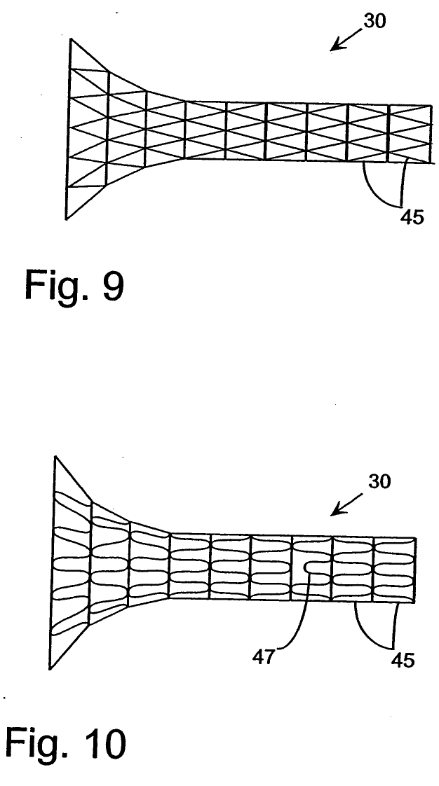

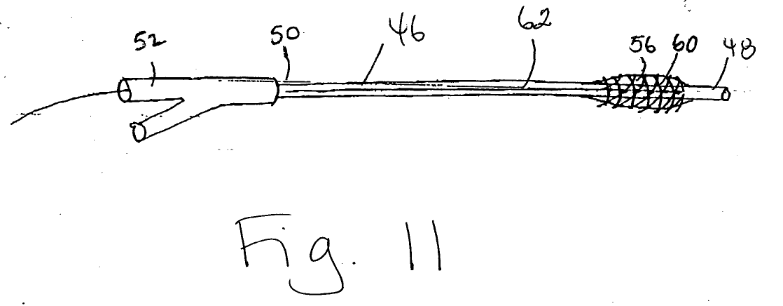

FIGS. 9 and 10 illustrate still further embodiments for the self-expanding shape memory stent 30. In FIG. 9, there is shown a stent having a plurality of annular sections 45 joined together to form the stent to the desired length. Each annular section is formed of a zigzag pattern of shape memory struts, which may be compressed when positioned in the catheter 20, and expand radially into contact with the pulmonary vein when deployed from the catheter. The proximal end of the stent shown in FIG. 9 may flare outward with larger diameter annular sections as shown so that the proximal end of the stent expands into contact with the atrial wall surrounding the pulmonary vein ostium when deployed from the catheter 20. It is understood that the proximal end may flare to a lesser or greater degree than shown in alternative embodiments.

In FIG. 10, there is shown a stent having a plurality of annular sections 45 joined together to form the stent to the desired length. Each annular section 45 is formed in a sinusoidal pattern of shape memory material, which may be compressed when positioned in the catheter 20, and which expands radially into contact with the pulmonary vein when deployed from the catheter. The proximal end of the stent shown in FIG. 10 may flare outward with larger diameter annular sections as shown so that the proximal end of the stent expands into contact with the atrial wall surrounding the pulmonary vein ostium when deployed from the catheter 20. It is understood that the proximal end may flare to a lesser or greater degree than shown in alternative embodiments. It is further contemplated that not all of the loops of adjacent annular sections be affixed, such as for example shown at loop 47 in FIG. 10.

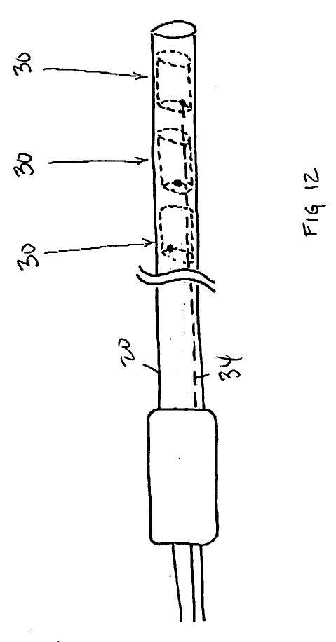

It is understood that the proximal ends of stents 30 shown in FIGS. 9 and 10 need not flare outward, in which embodiments, the proximal end of stent 30 would preferably reside within the pulmonary vein. It is further understood that, because the pulmonary veins may not be perfectly circular in cross section, but may alternatively have an oblong, oval or other shape cross section, the stent 30 according to the present invention may have a cross sectional shape provided to generally match that of the pulmonary vein to which the stent 30 is to be deployed. As an alternative to a self-expanding stent that is deployed from the distal tip of a catheter, a stent in accordance with the present invention may alternatively be deployed from a conventional balloon catheter, 4-12 French, made, for example, by Medtronic or Guidant. Such an embodiment is shown in FIG. 11. Catheters of other configurations and diameters are contemplated. As shown in FIG. 11, a catheter 46 includes a distal end 48 and a proximal end 50 having a handle 52 and controls 54 for manipulating distal end 48. Distal end 48 further includes an expandable (such as an angioplasty) balloon 56 about which is mounted a stent 60 for deployment in a pulmonary vein.

Stent 60 may be a helical stent as shown in FIG. 4, a double reverse helical stent as shown in FIG. 8, a plurality of affixed annular sections including zigzag struts as shown in FIG. 9 or a plurality of annular sections including sinusoidal shaped members as shown in FIG. 10. Other shapes are contemplated.

As described with respect to earlier embodiments, distal end 48 of catheter 46 may be positioned within a pulmonary vein through a transseptal sheath terminating in the left atrium. A contrast dye may be injected through a lumen in catheter 46 into the pulmonary vein to allow fluoroscopic visualization of the size and contours of the vein, as well as to ensure proper deployment of the stent 60.

Once properly positioned, actuation of one of the controls 54 may cause the balloon 56 to inflate, thereby expanding the stent 60. Balloon 56 inflates until the stent 60 lies in firm contact with the wall around the circumference of the pulmonary vein. Thereafter, the balloon is deflated via a control 54. For embodiments of the present invention including a flared proximal end of the stent, the balloon would have a larger diameter at its proximal end in comparison to distal portions of the balloon to ensure that the flared proximal portions of the stent are fully expanded.

As is known in the art, stent 60 in this embodiment is inelastic, and once expanded into contact with the pulmonary vein wall, and, possibly, the left atrial wall, the stent separates from the balloon and remains in contact with the pulmonary vein/atrial wall as the balloon deflates. The balloon 56 and catheter 46 may thereafter be withdrawn from the pulmonary vein.

Once the stent 60 is in contact with the pulmonary vein, RF energy may be applied to the stent via a wire 62 having a first end in contact with stent 60 and a second end attached to an RF generator as described above. Wire 62 may be a guide wire conventionally used to position the catheter in the left atrium. Alternatively, it may be a wire separate from the guide wire. The RF energy may be applied while the balloon is still inflated and/or after the balloon is deflated. Once the tissue has been ablated, the wire 62 may be detached from the stent according to mechanisms shown, for example, in FIGS. 5 through 7 described above or by other known detachment mechanisms.

As described in the Background of the Invention section, stenosis of the pulmonary vein is a significant concern in conventional RF catheter ablation procedures. However, in accordance with the present invention, after tissue ablation and removal of the catheter as described above, the stent 30/60 remains behind in position in the pulmonary vein or in the pulmonary vein and left atrium. The stent provides structural integrity to the ablated sections of the pulmonary vein to prevent post-procedure stenosis which may otherwise occur in conventional RF catheter ablation due to scar tissue or muscular contraction secondary to trauma.

In addition, the scar-causing action of delivering RF energy through the stent to the tissue causes the stent to adhere to the tissue, leaving the stent little or no chance of migrating after it has been deployed.

As discussed in the Background of the Invention section, conventional RF catheter ablation techniques can cause damage to surrounding extracardiac structures such as bronchioles, the right pulmonary artery and lung tissue, as well as causing thrombosis and/or embolism in the blood flowing through the pulmonary vein. It is a further feature of the present invention to significantly reduce or remove these risks.

In particular, the inner surface of stent 30/60 (i.e., the surface past which venal blood flows) may be coated with a thin, nonconductive polymer or other material to prevent the RF energy from passing from the stent to the blood and to direct all of the RF energy to the tissue which the stent contacts. Thus, the risk of thrombosis and embolism is minimized. The outer surface of the stent 30/60 (i.e., the surface in contact with the pulmonary vein wall) has no such insulative coating and is conductive to allow transmission of the RF energy to the pulmonary vein wall and/or the atrial wall. The outer and/or inner surface of stent 30/60 may however include an antineoplastic compound, such as for example tamoxifen, to inhibit cellular growth or intimal hyperplasia around the stent. The coating on the inner surface of the stent 30/60 may also include an anti-thrombotic, such as for example heparin, to further ensure that the blood passing through the stent does not clot. The coating may additionally or alternatively include antiarrhythmic medication or compound coatings such as amiodarone. The coating or the stent itself may further include a radioactive material to help prevent stenosis. Moreover, in embodiments where the stent is deployed by balloon catheter, the coating reduces the possibility of the stent edges rupturing the balloon.

As the stent 30/60 lies in firm contact with the walls of the pulmonary vein or pulmonary vein and atrium, ablation of the tissue may be accomplished with a relatively small amount of energy. In one embodiment, the energy may be 1 watt to 50 watts applied for 5 to 60 seconds, and more particularly 5 watts to 25 watts applied for 15 to 45 seconds. It is understood that the RF energy may be applied at wattages and times other than those set forth above in alternative embodiments. Thus, in addition to further reducing the risk of stenosis, thrombosis and embolism, the risk of damage to the surrounding tissues and structures is minimized.

It is known that AF may- be caused by aberrant cardiac cells in more than one of the pulmonary veins. It is therefore contemplated that a stent 30/60 as described above may be placed in more than one of the pulmonary veins during a procedure. This may be accomplished by deploying a first stent, withdrawing the catheter from the transseptal sheath, and introducing a new catheter containing another RF stent through the sheath to deploy the second stent in the next pulmonary vein. This process may be repeated to deploy stents in each of the four pulmonary veins to cure AF and prevent stenosis as described above. It is also contemplated that, after deploying a first stent, the delivery catheter remain positioned in the atrium. The pushwire may then be removed and reloaded with a new stent, which is then introduced through the catheter. In this embodiment, a new stent may be loaded onto the same pushwire, or a new pushwire and new stent may be used.

In a further alternative embodiment shown in FIG. 12, a single catheter may be pre-loaded with a plurality of stents 30. In such an embodiment, the catheter is positioned in a first pulmonary vein and a first stent is deployed. The pushwire 34 in this embodiment may include a distal mechanism capable of releasing and subsequently reattaching to a stent 30, such as for example the gripping jaws 36 shown in FIG. 5. Thus, after deployment of the first stent, the distal mechanism releases the first stent, moves proximally in the catheter to the second stent, and attaches to the second stent. This process may be performed by the cardiologist under x-ray fluoroscopy.

The catheter may then be repositioned in a second pulmonary vein and a second stent deployed. This process may be repeated to deploy stents in each of the pulmonary veins including ectopic cardiac cells that may be causing AF. It is contemplated that each of the pre-loaded stents of this embodiment be of the same size, or of different sizes, so that the cardiologist may deploy different sized stents in different sized pulmonary veins.

Although the invention has been described in detail herein, it should be understood that the invention is not limited to the embodiments herein disclosed. Various changes, substitutions and modifications may be made thereto by those skilled in the art without departing from the spirit or scope of the invention as described and defined by the appended claims.

I claim:

1. A stent for being placed in a pulmonary vein and the left atrium to treat atrial fibrillation by application of energy to the stent, and for preventing stenosis of the pulmonary vein, comprising:
a proximal end having a first circumferential size in an unbiased condition, and capable of being deployed in contact with the left atrium around the pulmonary vein ostium, said proximal end including loops, a portion of one loop in said proximal end lying in contact with at least a portion of the next adjacent loop of the stent to form a closed loop in the proximal end of the stent when deployed into contact with the left atrium surrounding the pulmonary vein ostium; and
a distal having a second circumferential size selected to fit snugly in contact with walls of the pulmonary vein in an unbiased condition, said second circumferential size being smaller than said first circumferential size of said proximal end, said distal end including loops, a portion of one loop in said distal end lying in contact with at least a portion of the next adjacent loop of the stent to form a closed loop in the distal end of the stent when deployed in contact with the pulmonary vein.

2. A stent as recited in claim 1, at least said loops in said proximal end of said stent including a biasing force for biasing said loops against each other, said biasing force capable of pulling said loops into contact with the atrial wall around the pulmonary vein ostium when said proximal end is deployed in the left atrium.

3. A stent as recited in claim 1, wherein an inner surface of said stent is coated with an antithrombotic compound to assist in blood clot prevention.

4. A stent as recited in claim 3, wherein said antithrombotic compound is heparin.

5. A stent as recited in claim 1, said stent formed of a self-expanding, shape memory material capable of naturally expanding into contact with the pulmonary vein and the left atrium around the pulmonary vein ostium.

6. A stent as recited in claim 5, wherein said self-expanding, shape memory material is nitinol.

7. A stent as recited in claim 1, wherein said stent is helically shaped.

8. A stent as recited in claim 1, wherein said stent is reverse double helically shaped.

9. A stent for being placed in a pulmonary vein and the left atrium to treat atrial fibrillation by application of energy to the stent, and for preventing stenosis of the pulmonary vein, comprising:
a distal end having a first cross sectional circumference for fitting snugly within the pulmonary vein: and
a proximal end have a second, flared cross sectional circumference larger than said first cross sectional circumference for fitting against the wall of the left atrium around the pulmonary vein ostium.

10. A stent as recited in claim 9, wherein said stent is capable of electrically isolating the pulmonary vein from the heart.

11. A stent as recited in claim 9, wherein said stent is capable of electrically isolating the pulmonary vein ostium from the heart.

12. A stent as recited in claim 9, wherein said stent is helical along its length.

13. A stent as recited in claim 9, wherein said stent is shaped to fit the cross sectional circumference of the pulmonary vein.

14. A stent as recited in claim 9, wherein said stent is formed of a shape memory material capable of being deployed from within a catheter.

15. A stent as recited in claim 9, wherein said stent is formed of a shape memory material capable of being deployed by an elastic balloon attached to a distal end of a catheter.

16. A stent as recited in claim 9, wherein said stent is comprised of a plurality of annular sections affixed to each other.

17. A stent as recited in claim 16, wherein said annular sections are comprised of electrically conductive struts in a zigzag pattern.

18. A stent as recited in claim 16, wherein said annular sections are comprised of electrically conductive material in a sinusoidal shaped pattern.
